(19) United States
(12) Patent Application Publication
(10) Pub. No.: US 2006/0182730 A1
(43) Pub. Date: Aug. 17, 2006

(54) Antiobesity agent using hen's egg antibody against digestive enzymes

(76) Inventors: Yoshikatsu Kodama, Gifu (JP); Hideo Goshima, Gifu (JP)

Correspondence Address: WENDEROTH, LIND & PONACK, L.L.P., 2033 K STREET N. W., SUITE 800, WASHINGTON, DC, 20006-1021, US

(21) Appl. No.: 10/552,062

(22) PCT Filed: Jul. 7, 2004
(86) PCT No.: PCT/JP04/05006

§ 371 Date: Oct. 5, 2005

(30) Foreign Application Priority Data
Apr. 10, 2003 (JP) 2003-106670

(51) Int. Cl. A61K 38/54; A61K 35/54; A61K 39/395

(52) U.S. Cl. 424/94.3; 424/581; 424/146.1

(57) ABSTRACT

Objects of the present invention are to provide an agent for inhibiting a digestive enzymes and an antiobesity agent having digestive-enzyme-inhibiting activity with high substrate specificity and having high safety. Provided is a composition comprising eggs produced by hens immunized with digestive enzymes or fragments thereof, or the processed products thereof, and containing 2 or more types of digestive enzymes.

### TECHNICAL FIELD

The present invention relates to an antibody having an effect of preventing and ameliorating obesity, a food and an antiobesity agent containing the antibody.

### BACKGROUND ART

Recently, obesity is increasing because of overnutrition and the like resulting from the westernization of dietary habits. Also among pet animals, obesity is increasing in a similar manner. Obesity is one of risk factors of arteriosclerosis, and is also involved in diabetes and hyperlipidemia, and as such it is a serious issue.

Obesity is a condition where fat is excessively accumulated in vivo. This occurs due to excessive intake of saccharides or fat. In the mechanism that leads to obesity as a result of excessive intake of saccharides, saccharides contained in ingesta are digested to result in monosaccharides and the monosaccharides are absorbed from the small intestine into the body to increase blood glucose values. In response to this stimulation, insulin acts on adipose cells to cause them to incorporate the monosaccharides in blood to convert them into fat. Furthermore, fat (triglyceride) with the most calories among food ingredients is hydrolyzed by pancreatic lipase into diacylglycerol and monoacylglycerol, thus resulting in glycerol and fatty acid that are absorbed from the small intestine. That is, excessive caloric intake acts so as to increase calories in the pancreas. Specifically, excessive fat intake leads to the development of obesity, or hyperlipidemia or arterialsclerosis.

Hence, it is expected that antiobesity action may be obtained by inhibiting a part of any of these pathways linked to obesity. Specifically, it is considered that obesity can be prevented or improved by action inhibiting glycolytic enzymes, action suppressing monosaccharide absorption, or action suppressing increases in blood glucose levels, which inhibit the pathway beginning with the excessive intake of saccharides and resulting in obesity. Similarly, it is also considered to be possible to prevent obesity by causing decreases in cholesterol levels and in blood triglyceride levels by lipolytic enzyme-inhibiting action that inhibits the pathway beginning with the excessive intake of fat and resulting in obesity.

The action inhibiting glycolytic enzymes, the action suppressing monossaccharide absorption, or the action suppressing increases in blood glucose levels inhibit the pathway leading to obesity as a result of the intake of saccharides.

An agent for inhibiting glycolytic enzymes inhibits glycolytic enzymes participating in the breakdown of polysaccharides to monosaccharides to delay the digestion of saccharides by ingestion, thereby suppressing acute increases in blood glucose levels after meals. When the functions of glycolytic enzymes are inhibited, breakdown of polysaccharides to monosaccharides occurs gradually, causing delayed absorption of monosaccharides from the small intestine and suppressed increases in blood glucose levels. It is thought that as a result, synthesis of lipids from saccharides is suppressed and the accumulation of body fat decreases.

Besides, since it is thought that acute increases in blood glucose levels and excessive secretion of insulin after meals because of excessive intake of saccharides also promote diabetes and hyperlipidemia in addition to obesity (see JAPANESE PHARMACOLOGY & THERAPEUTICS, vol. 19, No. 10, 284 (1991)), both diabetes and hyperlipidemia may be prevented or improved by inhibiting glycolytic enzymes. Moreover, preventing hyperlipidemia is effective for the prevention of arteriosclerosis. Accordingly, the agent for inhibiting glycolytic enzymes, the agent for inhibiting monosaccharide absorption, or the agent for suppressing increases in blood glucose levels is considered to be useful as an anti-diabetic agent or an anti-hyperlipidemic agent, and furthermore as an anti-arteriosclerosis agent.

An example of an agent for inhibiting glycolytic enzymes currently used as a pharmaceutical is α-glucosidase inhibitor, which has been confirmed to have an effect of suppressing increases in blood glucose levels after meals in animal experiments and clinical tests, and regarding which anti-obesity effects and anti-diabetes effects have also been reported (see Res. Exp. Med. 175: 87 (1979); Nippon Nogeikagaku Kaishi, vol. 63, 217 (1989); New Current 6: 2 (1995)).

Next, lipase-inhibiting action inhibits the pathway beginning with the intake of fat (triglycerides) and resulting in obesity. Fat is degraded by pancreatic lipase, and then absorbed from the small intestine. Thus, it is considered that an antiobesity effect can be obtained by inhibiting the enzyme activity of lipase so as to lower blood triglyceride levels. It is also considered that since suppression of fat absorption from the bowel leads to decreases in serum lipid levels, an agent for inhibiting lipase is useful as an anti-hyperlipemic agent.

Numerous chemically synthesized compounds and natural compounds having antiobesity action are known. When a chemically synthesized compound is administered, there may be concern about the problem of safety. In the meantime, even there is a need to incorporate an antiobesity agent into foods in the hope of preventing obesity in everyday life, discomfort is shown in many cases upon consumption because of its being a chemically synthesized compound due to large dosage amounts. To cope with such social needs, many antiobesity agents derived from natural products have mainly been developed.

For example, a considerable number of ingredients having antiobesity action such as hydroxycitric acid, nojirimycin, procyanidin, flavonoid and the glucosides thereof, catechins, hinokitiol, benzophenone derivatives, triterpene compounds and the derivatives thereof, sclerotiorin, caulerpenin, and coleusforskohli have been specified. These ingredients are used after purification from extracts of plants, marine algae, or the like, or used intact as extracts. Because of their low substrate specificity, the inhibitory action of these ingredients derived from natural products on digestive enzymes, and thus their antiobesity action, is unsatisfactory. Besides, there is concern that they cause side effects such as dyspepsia depending on their dosage.

We discovered that glucosyltransferase that is present in the extracellular membrane of Streptococcus mutans, the pathogen of dental caries, synthesizes saliva-insoluble glucan. We then discovered that a hen-egg antibody obtained by immunizing an egg-laying hen with the enzyme exhibits an anti-dental-caries effect by efficiently inactivating the enzyme activity thereby suppressing glucan synthesis (see JP Patent No. 2641228).

However, there has been no example wherein the enzyme is inhibited using an antibody against the above digestive enzyme. Furthermore, in preparation of an antibody using a hen, sufficient antibody titer cannot always be obtained depending on the type of antigen. Therefore, it has remained unknown whether or not a hen-egg antibody against a digestive enzyme such as a glycolytic enzyme, a lipolytic enzyme, or the like has sufficient antiobesity action by inhibiting enzyme activities within the small intestine.

### DISCLOSURE OF THE INVENTION

An object of the present invention is to provide an agent for inhibiting digestive enzymes and an antiobesity agent having digestive enzyme-inhibiting activity with high substrate specificity and high safety.

As a result of intensive studies to achieve the above object, we have discovered that the above problem can be solved by hen-egg antibodies prepared from eggs produced by hens immunized with digestive enzymes such as a glycolytic enzyme, a lypolytic enzyme, and the like, and thus we have completed the present invention.

We have confirmed that the above antibody significantly suppresses the activities of these digestive enzymes in vitro. We have also confirmed action suppressing increases in blood glucose levels, action suppressing glucose absorption, action lowering blood triglyceride levels, and action lowering cholesterol levels in animal experiments using saccharide (starch)-loaded or lipid (cone oil)-loaded rats. More surprisingly, we have confirmed that when a composition containing 2 or more types of antibodies is administered, a synergistic antiobesity effect can be obtained.

That is, the present invention encompasses the following inventions.

(1) A composition comprising an egg produced by a hen immunized with a digestive enzyme or a fragment thereof, or the processed product thereof, wherein the digestive enzyme comprises 2 or more types of digestive enzymes.

(2) The composition of (1), which comprises an egg produced by the same hen immunized with 2 or more types of digestive enzymes or fragments thereof, or a processed product thereof.

(3) The composition of (1), comprising a mixture of an egg produced by a hen immunized with at least 1 type of digestive enzyme or a fragment thereof, or the processed product thereof, and an egg produced by a hen immunized with a digestive enzyme differing from the digestive enzyme, a fragment thereof, or a processed product thereof.

(4) The composition of any one of (1) to (3), wherein the digestive enzyme is selected from the group consisting of a glycolytic enzyme, a lipolytic enzyme, and a proteolytic enzyme.

(5) The composition of any one of (1) to (4), wherein the processed product of the egg is an antibody.

(6) An agent for inhibiting a digestive enzyme, which comprises the composition of any one of (1) to (5).

(7) An antiobesity agent, which comprises the composition of any one of (1) to (5).

(8) A food, which contains the composition of any one of (1) to (5).

In the present invention, a digestive enzyme means an enzyme involved in digestion. Examples of a digestive enzyme that can be used as an immunogen in the present invention are not specifically limited, and include a glycolytic enzyme, a lipolytic enzyme, a proteolytic enzyme, and nuclease. A glycolytic enzyme, a lipolytic enzyme, and a proteolytic enzyme are preferably used.

In the present invention, a glycolytic enzyme means an enzyme having activity to degrade an oligosaccharide including a disaccharide or a polysaccharide as a substrate. Examples of a glycolytic enzyme used in the present invention are not specifically limited, and include polyase degrading a polysaccharide as a substrate and oligase degrading an oligosaccharide as a substrate. Examples of polyase include α-amylase, β-amylase, cellulase, and inulinase. Examples of oligase include α-glycosidase and β-glycosidase such as sucrase, maltase, isomaltase, lactase, and trehalase. In the present invention, amylase, specifically, pancreatic α-amylase, is preferably used.

In the present invention, a lipolytic enzyme means an enzyme having activity to degrade a neutral lipid or a phosphatide as a substrate. Examples of a lipolytic enzyme that can be used in the present invention are not specifically limited, and include lipase degrading a neutral lipid as a substrate and phospholipase degrading phospholipids as a substrate. In the present invention, pancreatic lipase is preferably used.

In the present invention, a proteolytic enzyme means hydrolase that acts on a protein as a substrate, and thus promotes the degradation of the peptide bond (—CO—NH—) thereof. Examples of a proteolytic enzyme that can be used in the present invention are not specifically limited, and include those acting on the internal peptide chain of a protein (that is, peptidyl peptide hydrolase), those acting on the terminus having an amino group of the peptide chain (aminoacyl peptide hydrolase) of a protein, those acting on the terminus having a carboxy-group of the internal peptide chain (peptidyl amino acid hydrolase) of a protein, and those acting on the further generated dipeptide (dipeptide hydrolase). Specific examples of the proteolytic enzyme include pepsin, trypsin, chymotrypsin, papain, collagenase, subtilisin, and carboxypeptidase. In the present invention, pepsin, particularly gastric pepsin, is preferably used.

In the present invention, it is preferred to use 2 or more types of digestive enzymes as immunogens, and a composition containing 2 or more types of antibodies against them. This is because a synergistic antiobesity effect can be obtained by combining antibodies against 2 or more types of digestive enzymes. Examples of combinations of digestive enzymes include a combination of a glycolytic enzyme and a proteolytic enzyme, that of a glycolytic enzyme and a lipolytic enzyme, that of a proteolytic enzyme and a lipolytic enzyme, and that of a glycolytic enzyme, a proteolytic enzyme, and a lypolytic enzyme. In particular, a combination of a glycolytic enzyme and a lipolytic enzyme is preferred. Furthermore, 2 or more types of digestive enzymes also mean, for example, a combination of 2 different types of enzymes (e.g., amylase and maltase) belonging to glycolytic enzymes. More specifically, a combination of α-amylase and α-glucosidase, that of pepsin and trypsin, that of lipase and phospholipase, that of α-amylase and lipase, that of α-amylase and pepsin, and that of pepsin and lipase are preferred.

In the present invention, a hen may be immunized with 2 or more types of digestive enzymes, or hens may be immunized respectively with 2 or more types of digestive enzymes, and then eggs produced by each hen or the processed product thereof may be mixed.

Origins of these enzymes are also not specifically limited, as long as the enzyme can act as an immunogen in a hen to be immunized. For example, digestive enzymes derived from animal species such as mammals and birds, and plant species such as fungi and bacteria can be used. A digestive enzyme derived from an animal, in particular, a pig, is preferably used.

In the present invention, not only the whole enzyme, but also a fragment thereof can be used. The term “fragment” is used regardless of particularly length, as long as it contains the amino acid sequence of a target protein.

In the present invention, hens are immunized with the above enzyme or a fragment thereof as an antigen, so as to obtain eggs containing an antibody against the enzyme. As an enzyme to be used herein, a commercial product is available, and it can be prepared by isolation and purification from a supply source using techniques known in the art. Alternatively, an enzyme and the fragment thereof can also be prepared by making microbes to produce them using genetic engineering techniques based on a known amino acid sequence thereof, followed by purification.

Fragments of an enzyme can be prepared as peptide fragments by general peptide synthesis or the like. Conventional means can be employed for chemical synthesis of peptides. Examples of such means include an azide method, an acid chloride method, an acid anhydride method, a mixed acid anhydride method, a DCC method, an active ester method, a carboimidzole method, and an oxidation-reduction method. Furthermore, peptides can be synthesized by either a solid-phase synthesis method or a liquid-phase synthesis method. In addition, in the present invention, peptides can also be synthesized using a commercial automatic peptide synthesizer (e.g., automatic peptide synthesizer PSSM-8 of Shimadzu Corporation).

A peptide fragment that may be appropriately used in the present invention can be determined in view of requirements, such as that it be located on the surface layer of a protein, it does not form a helix structure, or it not contain any simple sequence such as a repeat sequence. Moreover, since peptide sequences to be used for immunization may be very analogous to each other between mammals, immunization is preferably carried out after enhancing immunogenicity by binding a carrier protein known in the art such as KLH or BSA to the peptide sequence.

Hens are immunized with the enzyme or the fragment thereof prepared as described above as an antigen. Hens that are immunized herein are not specifically limited. In view of mass production of antibodies, egg-laying species, for example, white leghorn, are preferably used. Birds other than hens can also be immunized. If necessary, an adjuvant such as Freund's complete adjuvant (FCA) and Freund's incomplete adjuvant (FIA) can also be used. Immunization can be carried out mainly by intravenous, subcutaneous, intramuscular, or intraperitoneal injection, or can also be carried out by rhinenchysis, instillation, or the like. In addition, intervals for immunization are not specifically limited. Immunization is carried out 1 to 10 times at intervals of several days to several weeks. In general, several weeks after the initial immunization, antibodies reacting specifically to the administered antigens can be obtained in eggs and particularly in egg yolks.

Antibody titer in egg yolks can be measured using an enzyme-linked immunosorbent assay (ELISA), radioimmunoassay, or the like. After immunization, changes in antibody titer can be traced by measuring antibody titer at intervals of approximately 2 weeks. Generally, high antibody titers can be continuously obtained for approximately 3 months. In addition, when a decrease is observed in an antibody titer after immunization, the antibody titer can be increased by properly carrying out booster immunization at appropriate intervals.

In the present invention, for example, an agent for inhibiting enzyme or foods having antiobesity action, is produced using the above-immunized hens' eggs and the processed product thereof. In the present invention, examples of the processed product of eggs are not specifically limited, as long as they contain an antibody against a digestive enzyme used as an antigen for immunization of hens, and include whole eggs, egg yolks, and egg albumen of immunized hens, the egg liquid thereof, and extracts made from the egg liquid using propanol or chloroform. An egg yolk component is preferably included. Egg products powdered by a spray-dry method, a freeze-dry method, or the like are also included. Furthermore, egg products prepared by removing the yolk lipid component from egg yolks by a method using hydroxypropylmethylcellulose phthalate, polyethylene glycol, dextran sulfate, or the like and then powdering the resultant are also included. Furthermore, the processed products of eggs in the present invention also encompass an antibody itself that is purified from eggs and the above processed products of eggs by a known method such as ammonium sulfate salting out, sodium sulfate salting out, a low temperature ethanol precipitation method, ion exchange chromatography, gel filtration, and affinity chromatography. The thus prepared antibody is referred to as an hen-egg antibody. To enhance the preservation of the processed product, sterilized liquid whole egg or liquid egg yolk is preferably powdered by spray drying or freeze-drying.

The digestive-enzyme-inhibiting activity of the eggs and the processed products thereof of the present invention can be measured by a method known in the art, such as the Caraway method. When the activity was measured by the above method, it was revealed that the eggs and the processed products thereof of the present invention containing antibodies against digestive enzymes possess significant enzyme-inhibiting activity.

Therefore, foods having an antiobesity effect can be produced by incorporating the eggs and the processed products thereof of the present invention into foods and health foods as food additive ingredients. Examples of foods for which the eggs and the processed products thereof of the present invention are used are not specifically limited, and include foods containing eggs produced by a general production method. For example, yogurt, pudding, ice cream, candy, gum, and mayonnaise are preferred. They are preferably used in the production of health foods having an antiobesity effect. When they are incorporated into general foods, in the case of a powdered active ingredient, incorporation of 0.001% to 15% by weight, and in particular, 0.1% to 5% by weight of a food is preferred. The powdered ingredient can be incorporated in amounts less than or more than those constituting the above range depending on the types of foods.

Moreover, the antibody against a digestive enzyme of the present invention can be used in the production of a pharmaceutical composition having an effect of inhibiting the digestion of saccharides, protein, and lipids, because it has activity to inhibit the activity of the digestive enzyme. By the use of an antibody against a glycolytic enzyme, an agent for inhibiting a glycolytic enzyme, an agent for inhibiting saccharide absorption, an agent for suppressing increases in blood glucose levels, or the like can be produced. Furthermore, by the use of an antibody against a lipolytic enzyme, an agent for inhibiting a lipolytic enzyme, an anti-hyperlipidemic agent, an agent for lowering blood triglyceride levels, an agent for lowering cholesterol levels, or the like can be produced. Furthermore, by the use of an antibody against a proteolytic enzyme, an agent for inhibiting a proteolytic enzyme, an agent for suppressing hyperproteinemia, or the like can be produced. In addition, all of these preparations, that is, each thereof and combinations thereof have digestive enzyme-inhibiting activity, and thus have antiobesity action. In this specification, antiobesity action means action preventing excessive in vivo accumulation of fat, and action decreasing excessively accumulated fat.

The eggs containing antibodies against the enzymes of the present invention and the processed products thereof containing the antibodies can be formulated into oral preparations intact or together with commonly used additives in the form of, for example, tablets, granules, powders, capsules, and liquid drugs by a general formulation method. Examples of additives include excipients, binders, disintegrating agents, lubricants, anti-oxidants, coloring agents, and flavoring agents, and they are used as necessary. To enable sustained release of an agent so that it can act for a long time at the small intestine site, coating can be done using a known retarder or the like. As an excipient, for example, sodium carboxymethylcellulose, agar, light anhydrous silicic acid, gelatine, crystalline cellulose, sorbitol, talc, dextrin, starch, lactose, saccharose, glucose, mannitol, magnesium aluminometasilicate, or calcium hydrogenphosphate can be used. Examples of a binder include gum Arabic, sodium alginate, ethanol, ethyl cellulose, sodium casein, sodium carboxymethylcellulose, agar, purified water, gelatine, starch, gum tragacanth, lactose, hydroxycellulose, hydroxymethylcellulose, hydroxypropylcellulose, and polyvinylpyrrolidone. Examples of a disintegrating agent include carboxymetylcellulose, sodium carboxymethylcellulose, calcium carboxymetylcellulose, crystalline cellulose, starch and hydroxypropyl starch. Examples of a lubricant include stearic acid, calcium stearate, magnesium stearate, talc, hardened oil, sucrose fatty acid ester, and waxes. Examples of an antioxidant include tocopherol, gallic acid ester, dibutyl hydroxytoluene (BHT), butylhydroxyanisol (BHA), and ascorbic acid. Furthermore, if necessary, other additives or drugs, for example, antacids (e.g., sodium hydrogen carbonate, magnesium carbonate, precipitated calcium carbonate and synthetic hydrotalcite), or gastric mucosa protective agents (e.g., synthetic aluminum silicate, sucralfate and sodium copper chlorophyllin) may be added.

Examples of objects to which preparations, such as the above agent for inhibiting a digestive enzyme, an antiobesity agent, or the like can be administered are not specifically limited, as long as they are animals having digestive enzymes that can act as antigens against antibodies contained in these preparations, and include mammals and birds. In particular, the above preparations can be used for humans and pet animals, and can be appropriately used for, for example, dogs and cats.

### BRIEF DESCRIPTION OF THE DRAWINGS

FIG. 1 shows blood glucose contents in rats to which an anti-pancreatic amylase antibody purified from hen-egg yolks or an antibody purified from the egg yolks of unimmunized hens was administered in Example 3.

FIG. 2 shows blood insulin contents in starch-loaded rats to which the anti-pancreatic amylase antibody purified from hen-egg yolks or the antibody purified from the egg yolks of unimmunized hens was administered in Example 3.

FIG. 3 shows plasma triglyceride contents in the blood of rats to which a fat solution containing the anti-pancreatic lipase antibody purified from hen-egg yolks or a fat solution containing the antibody purified from the egg yolks of unimmunized hens was administered in Example 6.

FIG. 4 shows body weight gain in rat groups to which the anti-pancreatic amylase antibody purified from the hen-egg yolks alone was administered, the anti-pancreatic lipase antibody purified from the hen-egg yolks alone was administered, a mixture of the two antibodies was administered in equivalent amounts, and an antibody purified from the egg yolks of unimmunized hens was administered, respectively, by mixing the aforementioned substances with their feed in Example 7.

This specification includes the contents as disclosed in the specification of Japanese Patent Application No. 2003-106670, which is a priority document of the present application.

### BEST MODE FOR CARRYING OUT THE INVENTION

### EXAMPLE 1

### Preparation of Anti-Glycolytic Enzyme Hen-Egg Antibody

(1) Swine Pancreatic Amylase

In this example, α-amylase purified from swine pancreas was used as a glycolytic enzyme. Antigens for immunization, immobilized antigens for ELISA, and swine pancreatic amylase used in the examples were obtained from ELASTIN PRODUCTS CO., INC. (Missouri, U.S.A.). The enzyme activity of the swine pancreatic amylase used herein was 1,5000 units/mg protein.

(2) Production of Anti-Pancreatic Amylase Hen-Egg Antibody

As hens to be immunized, a group of around 18-week-old White Leghorn strain, Hyline W77 line hens was used. Swine pancreatic amylase obtained in (1) was adjusted to be 0.5 mg/mL (7500 units/mL), and then admixed with an oil adjuvant. 0.5 mL each of the mixture was injected into the pectoral muscle on the right and that on the left (initial immunization). 8 weeks later, booster immunization was carried out using an antigen (1.0 mg/mL (15,000 units/mL)) in an amount double that of the antigen used in the initial immunization. Antibody titer in the egg yolks produced by immunized hens was measured. From 2 weeks after booster immunization, at which time the immune titer significantly increased and became stable, the collection of eggs was begun, and continued for 4 weeks. In addition, the antibody titer in the egg yolks remained stable for 4 to 6 months. Subsequently, when the antibody titer decreased, injection was carried out in a manner similar to that of booster immunization, and it recovered to the original antibody titer level. In addition, the antibody titer in the hen eggs was measured by the following methods.

(3) Measurement of Antibody Titer of Anti-Pancreatic Amylase Antibody in Hen Egg Yolks

Egg yolks were removed from the immunized eggs by breaking the eggs, and were weighed. PBS in an equivalent amount was added to the yolks, and then the components of yolk liquid were dissolved well. To the mixture, an equivalent amount of chloroform was added, the mixture was shaken and agitated violently, and then centrifugation was carried out to obtain the supernatant. This supernatant was used as a sample for measuring antibody titer. Antibody titer was measured by ELISA. The method is as described below. Cross-titer was measured for the immobilized antigen (swine pancreatic amylase) and alkaline phosphatase-labeled anti-fowl IgG complex, so as to set an optimum concentration. As a plate, a 96-well Immulon 2 plate (Dynex) was used, and swine pancreatic amylase was used for immobilization. The antigen was diluted in a carbonic acid buffer (pH 9.6) to achieve a protein level of 5.0 μg/mL. 50 μL of solution was added per well, and then it was allowed to stand at +4° C. for 18 hours. When used, each well was washed 3 times with PBS-Tween, and 150 μL of 3.0% BSA solution was added to each well for blocking, and then it was allowed to stand at 37° C. for 60 minutes. Next, after each well had been washed 3 times with PBS-Tween, each sample was added in amounts of 50 μL per well, and then allowed to react at 37° C. for 60 minutes. After reaction, each well was washed again with PBS-Tween, 50 μL of alkaline phosphatase-labeled anti-fowl IgG complex diluted 2,000 times was added per well, and it was allowed to react again at 37° C. for 60 minutes. After the wells had been washed 5 times, a substrate (p-nitrophenyl phosphate) was added to cause color development at 37° C. 15 minutes later, 50 μL of a reaction stop solution (2M NaOH) was added per well to stop the reaction. Subsequently, absorbance (410 nm) of each well was measured using an ELISA autoreader. The antibody titers of the samples were finally calculated by correction using absorbances of the positive and the negative controls as standards.

(4) Preparation of Antibody Purified from Hen Egg Yolks

Immunized eggs were washed with water and then disinfected. Egg yolks were separated with an egg-breaking machine, subdivided in amounts of 8.0 kg each, and then stored until use at −20° C. or less. Purification was carried out by a method as described below. Specifically, 7.5 kg of egg yolks was used as a starting material, and purified water in a quantity 10 times greater than that of the yolk weight was added to remove fat. Ammonium sulfate was added to the supernatant to achieve 40% saturation, the mixture was agitated, and then pellets were obtained by centrifugation. The pellets were dissolved in physiological saline, and then the pellets were obtained again by 30% saturation salting out. These pellets were dissolved in a small volume of physiological saline, to which ethanol cooled to −20° C. was gradually added to achieve a final concentration of 50% while agitating the solution. After centrifugation, the pellets were dissolved in physiological saline and then freeze-dried. As a result, 11 g of light yellowish white powder was obtained. The recovery rate of the antibody was around 47%, the IgG purity was 95% or more, and the water content was 2.0% or less. In addition, the following examples were carried out using this anti-pancreatic amylase antibody purified from hen egg yolks. Moreover, an antibody purified from the egg yolks of unimmunized hens was obtained by a similar treatment from hen eggs obtained from the unimmunized hens, and this antibody was used as a negative control in the following examples.

### EXAMPLE 2

### Amylase Activity Inhibition Test

The inhibition rate of amylase activity was measured using human pancreatic α-amylase (ELASTIN PRODUCTS CO., INC. Missouri, U.S.A.) and “Amylase-Test Wako” manufactured by Wako Pure Chemical Industries. The anti-pancreatic amylase antibody purified from hen egg yolks and the solution of the antibody purified from the egg yolks of unimmunized hens and an enzyme solution (0.05 mg/mL human pancreatic amylase) were mixed in equivalent amounts. In addition, as a positive control, a buffer containing no antibodies and an enzyme solution were treated similarly and then used. Subsequently, the enzyme activities of these samples were measured using the “Amylase-Test Wako.” A method used herein involves preheating 1.0 mL of a substrate buffer (0.25 M phosphate buffer (pH 7.0) and 400 μg/mL soluble starch) at 37° C. for 5 minutes, adding 20 μL of the above mixed solution, and carrying out reaction at 37° C. for 7 minutes 30 seconds. Afterwards, 1.0 mL of a color development solution (0.01 N iodine solution) was added, and then 5.0 mL of distilled water was added. As a negative control, distilled water was used instead of a mixed solution. Absorbance of each sample was measured at wavelength of 660 nm. The inhibition rate of amylase activity was calculated using the following equation.
Amylase activity inhibition rate=[1−(AC−AT)/(AC−AP)]×100
AT: Absorbance of sample
AP: Absorbance of positive control
AC: Absorbance of negative control
Results

50% inhibition concentration (IC₅₀) of each antibody against the enzyme activity of amylase is shown in Table 1.

TABLE 1

| | IC₅₀ (mg/mL) |
|---|---|
| Anti-pancreatic amylase hen-egg antibody | 0.003 |
| Hen-egg antibody of unimmunized hens | >1.0 |

The anti-pancreatic amylase antibody in Table 1 has a superior effect of inhibiting amylase activity. The above antibody exhibits activity-inhibiting action against pancreatic amylase, the digestive enzyme being responsible for in vivo digestion and absorption of saccharides and being crucial regarding the problem of diabetes. This antibody can contribute to the suppression and prevention of diabetes and obesity by suppressing in vivo accumulation of saccharides.

### EXAMPLE 3

### Starch Loading Test

20 approximately 6-week-old Wister male rats that had been loaded with starch were divided into 2 groups. To the test group, 1.0 mL of 1.0% solution of the anti-pancreatic amylase antibody purified from hen-egg yolks obtained in Example 1(4) was administered once, and to the control group, the same amount of the antibody purified from the egg yolks of unimmunized hens was orally and forcibly administered. Blood was collected at 1 hour, 2 hours, and 3 hours after administration, and then blood glucose content and insulin content were measured.

Results

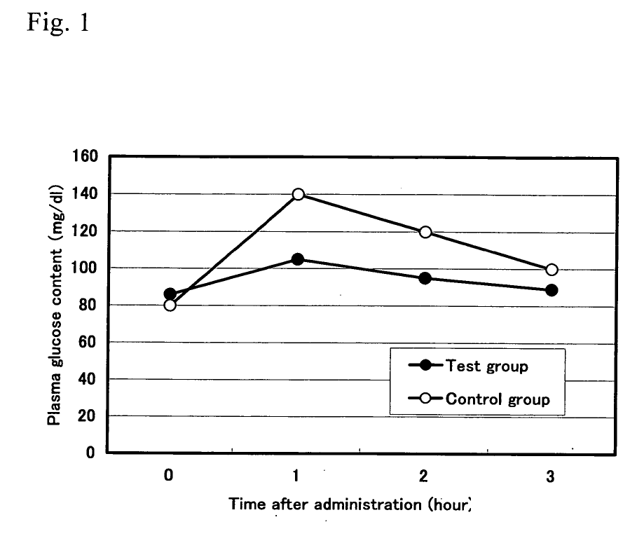

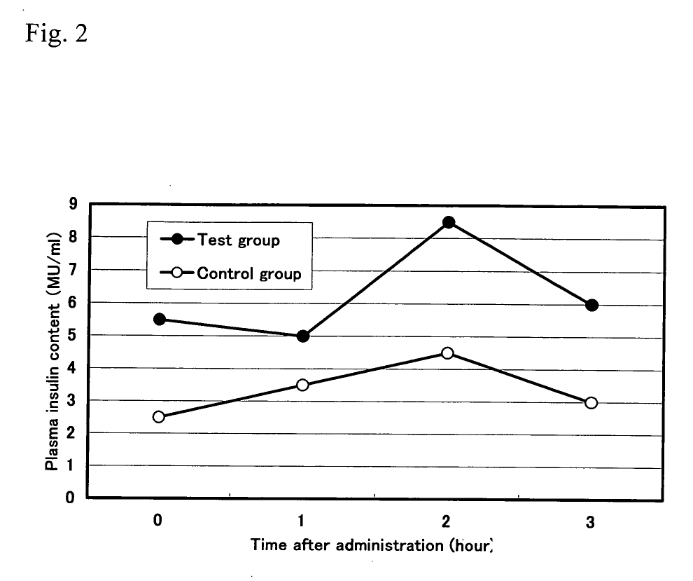

Experimental results are shown in FIGS. 1 and 2. The above results showed that, compared with the control group, the blood glucose concentration and insulin concentration were significantly lower in the group to which the antibody of the present invention inhibiting the enzyme activity of α-amylase and purified from the hen-egg yolks had been administered. Based on these results, it was concluded that the antibody of the present invention inhibits the functions of the digestive enzyme so as to limit the incorporation of carbohydrates into a body and to suppress increases in blood glucose levels. As a result, the antibody is expected to have applications regarding pharmaceuticals or health foods targeting antiobesity or antidiabetes.

### EXAMPLE 4

### Preparation of Anti-Pancreatic-Lipase Hen-Egg Antibody

(1) Pancreatic Lipase

Lipase purified from swine pancreas was used as pancreatic lipase. The antigen for immunization, an immobilized antigen for ELISA and swine pancreatic lipase used in this example were obtained from ELASTIN PRODUCTS CO., INC. (Missouri, USA). The enzyme activity was 45,000 units/mg protein.

(2) Production of Anti-Pancreatic-Lipase Hen-Egg Antibody

For immunization, a group of around 18-week-old White Leghorn species, Hyline W77 line hens was used. The swine pancreatic lipase obtained in (1) was adjusted to be 0.5 mg/mL (22,500 units/mL), and then admixed with an oil adjuvant. 0.5 mL each of the mixture was injected into the pectoral muscle on the left and that on the right (initial immunization). 8 weeks later, booster immunization was carried out using an antigen in an amount (1.0 mg/mL (45,000 units/mL)) double that of the antigen used in the initial immunization. From 2 weeks after booster immunization, at which time the antibody titer in egg yolks significantly increased and became stable, the collection of eggs was begun, and continued for 4 weeks. In addition, the antibody titer in the egg yolks remained stable for 4 to 6 months. Subsequently, when the antibody titer decreased, injection was carried out in a manner similar to that of booster immunization, and it recovered to the original antibody titer level.

(3) Measurement of Antibody Titer of Anti-Pancreatic Lipase Antibody in Hen-Egg Yolks

Egg yolks were removed from the immunized eggs by breaking the eggs, and weighed. PBS was added in an equivalent amount to the egg yolks, and then the components of the liquid egg yolk were dissolved well. To the mixture, an equivalent amount of chloroform was added, the mixture was shaken and agitated violently, and then centrifugation was carried out to obtain the supernatant. This supernatant was used as a sample for measuring antibody titer. Antibody titer was measured by ELISA. The method is as described below. Cross-titer was measured for the immobilized antigen (swine pancreatic lipase) and alkaline phosphatase-labeled anti-fowl IgG complex, so as to set an optimum concentration. As a plate, a 96-well Immulon 2 plate (Dynex) was used, and swine pancreatic lipase was used for immobilization. Antigen was diluted in a carbonate buffer (pH 9.6) to achieve a protein level of 5.0 μg/mL. 50 μL of the solution was added per well, and then it was allowed to stand at +4° C. for 18 hours. When used, each well was washed 3 times with PBS-Tween, 150 μL each of 3.0% BSA solution was added to each well for blocking, and then it was allowed to stand at 37° C. for 60 minutes. Next, after each well had been washed 3 times with PBS-Tween, each sample was added in amounts of 50 μL per well, and then allowed to react at 37° C. for 60 minutes. After reaction, each well was washed again with PBS-Tween, 50 μL of alkaline phosphatase-labeled anti-fowl IgG complex diluted 2,000 times was added per well, and then it was allowed to react again at 37° C. for 60 minutes. After the wells had been washed 5 times, a substrate (p-nitrophenyl phosphate) was added to cause color development at 37° C. 15 minutes later, 50 μL of a reaction stop solution (2M NaOH) was added per well to stop the reaction. Subsequently, absorbance (410 nm) of each well was measured using an ELISA autoreader. The antibody titers of the samples were finally calculated by correction using absorbances of positive and negative controls as standards.

(4) Preparation of Anti-Pancreatic Lipase Antibody Purified from Hen Eggs

Immunized eggs were washed with water and then disinfected. Egg yolks were separated with an egg-breaking machine, subdivided in amounts of to 8.0 kg each, and then stored until use at −20° C. or less. Purification was carried out by a method as described below. Specifically, 7.5 kg of egg yolks was used as a starting material, and purified water in a quantity 10 times greater than that of the egg yolk weight was added to remove fat. Ammonium sulfate was added to the supernatant to achieve 40% saturation, followed by agitation and centrifugation so as to obtain pellets. The pellets were dissolved in physiological saline, and then pellets were obtained again by 30% saturation salting out. These pellets were dissolved in a small amount of physiological saline, to which ethanol cooled to −20° C. was gradually added to achieve a final concentration of 50% while agitating the solution. After centrifugation, the pellets were dissolved in physiological saline and then freeze-dried. As a result, 11 g of light yellowish white powder was obtained. The recovery rate of the antibody was around 47%, the IgG purity was 95% or more, and the water content was 2.0% or less. In addition, the following examples were carried out using this anti-pancreatic lipase antibody purified from hen-egg yolks. Moreover, an antibody purified from the egg yolks of unimmunized hens was obtained from the hen eggs obtained from the unimmunized hens by a similar treatment, and this antibody was used as a negative control in the following examples.

### EXAMPLE 5

### Lipase Activity Inhibition Test

The inhibition rate of lipase activity was measured using human pancreatic lipase (ELASTIN PRODUCTS CO., INC. Missouri, U.S.A.) and “Lipase Kit S” manufactured by DAINIPPON PHARMACEUTICAL. A solution of the anti-pancreatic lipase antibody purified from hen-egg yolks and that of the antibody purified from the egg yolks of unimmunized hens were each mixed in equivalent amounts with an enzyme solution (0.05 mg/mL human pancreatic lipase). In addition, as a positive control, a buffer containing no antibodies and an enzyme solution, and as a negative control, distilled water, were treated similarly and then used. Subsequently, the enzyme activities of these samples were measured using the “Lipase Kit S.” A method used herein involves adding 100 μL of the mixed solution to 1 mL of a color development solution (a buffer containing 0.1 mg/mL 5,5′-dithiobis (2-nitrobenzoic acid (DTNB)), adding 20 μL of an esterase inhibitor (3.48 mg/mL phenylmethylsulfonylfluoride (PMSF)), and then admixing them. These were preheated at 30° C. for 5 minutes, 100 μL of a substrate solution (6.69 mg/mL dimercaprol tributyrate (BALB)+5.73 mg/mL sodium dodecyl sulfate (SDS)) was added and admixed therewith, and then the solution was allowed to react to 30° for 30 minutes while shielding it from light. Subsequently, 2.0 mL of a reaction stop solution was added to stop the reaction. To perform a blank run, each sample, the color development solution and the esterase inhibitor were mixed, and then the mixed solution was allowed to react at 30° C. for 5 minutes and then at 30° C. for 30 minutes, the reaction stop solution was added, and then the substrate solution was added. Absorbance of each sample was measured at a wavelength of 410 nm. The rate of the inhibition of lipase activity was calculated using the following equation.
Lipase activity inhibition rate=[1−(AC−ABt)/(AC−ABc)]×100
AT: Absorbance of sample
ABt: Blank absorbance of sample
AC: Absorbance of negative control
ABc: Blank absorbance of negative control
   Results

50% inhibition concentration (IC₅₀) of each antibody against the enzyme activity of lipase is shown in Table 2. As shown in Table 2, the anti-pancreatic-lipase antibody purified from hen-egg yolks has an excellent effect of inhibiting lipase activity. This antibody exhibits enzyme-activity-inhibiting action against pancreatic lipase, the digestive enzyme being responsible for in vivo digestion and absorption of lipids and being crucial regarding the problems of diseases such as hyperlipidemia accompanying obesity. This antibody can contribute to the prevention of these diseases by suppressing the in vivo accumulation of lipids.

TABLE 2

| | IC₅₀ (mg/mL) |
|---|---|
| Anti-pancreatic-lipase antibody purified from hen-egg | 0.001 |
| yolks | |
| Antibody purified from the egg yolks of unimmunized | >1.0 |
| hens | |

### EXAMPLE 6

### Fat-Absorption-Inhibiting Action Test

The anti-pancreatic-lipase antibody purified from hen-egg yolks and the antibody purified from the egg yolks of unimmunized hens obtained in Example 4 (4) were dissolved at a concentration of 10 mg/mL in corn oil, and then subjected to ultrasonication, thereby obtaining a test solution and a control solution for each antibody. The pancreatic lipase, which is a digestive enzyme, acts on the oil droplets (micell) formed by fat in foods with bile acids and phospholipids, so as to decompose and absorb fat. Thus, as a substrate solution corresponding to the droplet ingredient, a solution comprising the following composition of ingredients including corn oil as a main ingredient was ultrasonicated, so that a desired lipid solution was prepared.

TABLE 3
Composition of lipid solution

| Ingredients | Content | |
|---|---|---|
| Corn oil | 6.0 | mL |
| Cholesterol oleate | 2.0 | g |
| Cholic acid | 80 | mg |
| Purified water | 6.0 | mL |

Approximately 6-week-old Wister male rats were divided into 2 groups with 10 rats/group. To the test group, 1.0 mL of the fat solution containing the anti-pancreatic lipase antibody purified from hen-egg yolks was orally and forcibly administered. To the control group, the same volume of the fat solution containing the antibody purified from the egg yolks of unimmunized hens was orally and forcibly administered. After administration, blood was collected with passage of time. Plasma triglyceride values in blood were measured using a lipase kit S manufactured by DAINIPPON PHARMACEUTICAL, thereby demonstrating effects of suppressing fat absorption from the bowel.

Results

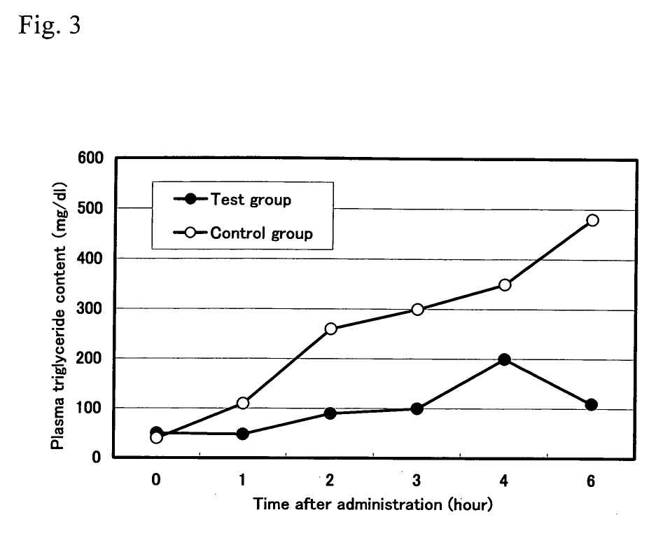

FIG. 3 shows the thus obtained fat-absorption-suppressing effects. In all the groups to which the fat solution containing the anti-pancreatic-lipase antibody purified from hen-egg yolks had been administered, increases in the plasma triglyceride values due to loading with corn oil were suppressed. These results revealed that the oral administration of the antibody purified from hen egg yolks of the present invention inhibiting the enzyme activity of pancreatic lipase significantly suppresses fat absorption.

### EXAMPLE 7

### Obesity-Suppressing Effect of Anti-Pancreatic-Amylase Antibody Purified from Hen-Egg Yolks and Anti-Pancreatic-Lipase Antibody Purified from Hen-Egg Yolks and Their Synergistic Effects

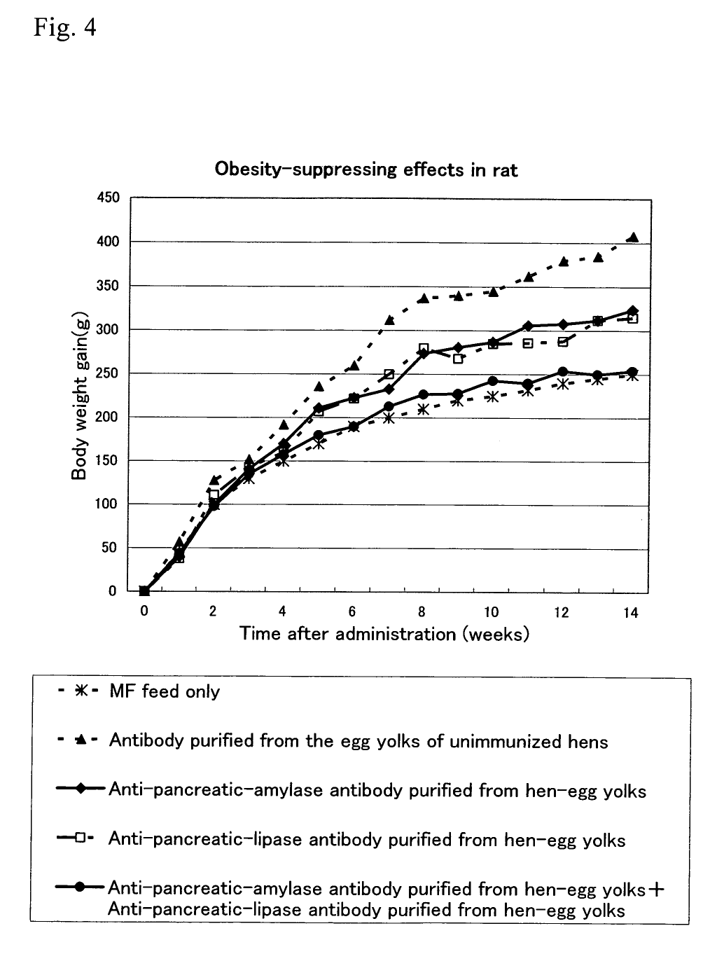

An obesity suppression test was carried out using the anti-pancreatic-amylase antibody purified from hen-egg yolks and the anti-pancreatic-lipase antibody purified from hen-egg yolks obtained in Example 1 (4) and Example 4 (4). In this test, 80 approximately 6-week-old Wister male rats were used. MF feed (powder) (Oriental Yeast) was mixed with corn oil and starch at a concentration of 10%, and fed ad libitum. Test groups were established to consist of 20 rats per group, to which 0.1% anti-pancreatic-amylase antibody purified from hen-egg yolks, 0.1% anti-pancreatic-lipase antibody purified from hen-egg yolks, and a mixture of 0.05% of each of the two antibodies were administered, respectively, by mixing the substances with their feed. Furthermore, a positive control group was similarly treated using an antibody purified from the egg yolks of unimmunized hens. Furthermore, a negative control group was developed by the feeding of only MF feed supplemented with neither corn oil nor antibody. The test period was 14 weeks, and then body weights were determined. FIG. 4 shows the results.

Results

Compared with the control group, body weight gain was suppressed in all the 3 groups to which the antibodies had been administered. However, in the group to which the mixture of the anti-pancreatic-amylase antibody purified from hen-egg yolks and the anti-pancreatic-lipase antibody purified from hen-egg yolks had been administered, body weight gain was suppressed even when compared with the 2 groups to which the anti-pancreatic-amylase antibody or the anti-pancreatic-lipase antibody had been singly administered. Thus, the combined use of the antibodies was confirmed to provide significant suppression of body weight gain.

### EXAMPLE 8

### Safety Test of Anti-Pancreatic-Amylase Hen-Egg Antibody and Anti-Pancreatic-Lipase Hen-Egg Antibody (Single Dose Toxicity Test)

A single dose toxicity test was carried out using 6-week-old F344/DuCrj female and male rats according to the Guidelines for Toxicity Studies of Drugs (“Good Laboratory Practice (GLP) ordinance specifying standards for implementation of non-clinical studies on Safety of Drugs” (Ordinance No. 21 of the the Ministry of Health and Welfare (MHW) dated Mar. 26, 1997)). Specifically, the anti-pancreatic-amylase antibody purified from hen eggs and the anti-pancreatic-lipase antibody purified from hen eggs prepared, respectively, in Example 1(4) and Example 2(4), and the two antibodies mixed in the same proportions by quantity, were suspended in physiological saline at 200 mg/mL each. The suspensions were variously, forcibly and orally administered at 2,000 mg/body weight kg, which is the maximum dosage according to the above guidelines, to the rats, and then the rats were observed for 7 days. As a result, no cases of death were observed in any of the groups, and no abnormalities were observed in terms of clinical symptoms or body weights. Moreover, no abnormalities were observed in a pathological test. Thus, the anti-pancreatic-amylase hen-egg antibody and the anti-pancreatic-lipase hen-egg antibody used in the present invention were confirmed to have extremely high safety when used independently or in combination.

### EXAMPLE 9

### Safety Test of Anti-Pancreatic-Amylase Antibody Purified from Hen-Egg Yolks and Anti-Pancreatic-Lipase Antibody Purified from Hen-Egg Yolks (Single Dose Toxicity Test)

A single dose toxicity test was carried out using 5-week-old ICR/Crj male mice of 29 to 32 g in body weight (5 mice per group) according to the Guidelines for Toxicity Studies of Drugs (Notification No. 118 of the Evaluation and Registration Division, Pharmaceutical Affairs Bureau (PAB) dated Feb. 15, 1984; To the Director of Each Prefectural Government Public Health Bureau; From the Director of the Second Evaluation and Registration Division, PAB, MHW.”). Specifically, the anti-pancreatic-amylase antibody purified from hen-egg yolks and the anti-pancreatic-lipase antibody purified from hen-egg yolks prepared, respectively, in Example 1(4) and Example 4(4), and the two antibodies mixed in the same proportions by quantity, were suspended in physiological saline at 30 mg/mL each. The suspensions were variously, forcibly and orally administered to mice at doses of 0.5 mL per 30 g in body weight (500 mg/body weight kg), and then the mice were observed for 14 days. As a result, no dead animals were observed in any of the groups or cases, no side effects were observed, and no microscopic abnormalities were observed in the tissues and organs by autopsy on day 14. Thus, it was shown that the anti-pancreatic-amylase antibody purified from hen-egg yolks and the anti-pancreatic-lipase antibody purified from hen-egg yolks used in the present invention have extremely low toxicity when used independently or in combination.

Production example 1 Ice cream

| | Prescription A | Prescription B |
|---|---|---|
| Salt-free butter | 7.0% | 7.0% |
| Whole milk condensed milk | 10.0% | 10.0% |
| Milk | 35.0% | 35.0% |
| Skim milk | 0.5% | 3.0% |
| Granulated sugar | 4.0% | 4.0% |
| 75% Brix starch syrup | 14.0% | 14.0% |
| Emulsion stabilizer | 0.5% | 0.5% |
| Liquid egg yolk of the | 3.0% | — |
| present invention | | |
| Liquid whole egg of the | — | 0.5% |
| present invention | | |
| Water | 26.0% | 26.0% |
| Aromatic | Proper quantity | Proper quantity |

Production example 2 Candy

| | |
|---|---|
| Frozen concentration mandarin orange juice | 5.0% |
| Fructose glucose liquid sugar | 11.0% |
| Citric acid | 0.2% |
| L-ascorbic acid | 0.02% |
| Aromatic | 0.2% |
| Coloring matter | 0.1% |
| Powdered whole egg of the present invention | 0.2% |
| Water | 83.28% |

Production example 3 Chocolate

| | |
|---|---|
| Chocolate | 45.0% |
| Sucrose | 15.0% |
| Cacao butter | 20.0% |
| Whole milk powder | 19.9% |
| Powdered whole egg of the present invention | 0.1% |

Production example 4 Beverage

| | | |
|---|---|---|
| Defatted and powdered whole egg of the present invention | 1 | g |
| Xylitol | 10 | g |
| Vitamin B₁ hydrochloride | 0.5 | mg |
| Vitamin B₂ | 0.2 | mg |
| Vitamin C | 500 | mg |
| Niacin | 1.0 | g |
| Calcium pantothenate | 0.2 | mg |
| Water | 100 | ml |

Production example 5 Tablet

| | | |
|---|---|---|
| Coat calcium | 108 | g |
| Ferric pyrophosphate | 2 | g |
| Ascorbic acid | 40 | g |
| Microcrystalline cellulose | 40 | g |
| Reduced malt sugar | 285 | g |
| Powdered whole egg of the present invention | 0.5 | g |

Tablets were made after admixture.

Production example 6 Dried soup

| | |
|---|---|
| Hen egg | 3.6 g |
| Meat extract | 1.0 g |
| Onion extract | 1.7 g |
| Carrot paste | 2.1 g |
| Kombu extract | 0.1 g |
| Emulsifier | 0.1 g |
| Table salt | 0.2 g |
| Aromatic (red pepper) | 0.2 g |
| Seasoning (e.g., amino acid) | 0.2 g |
| Powdered whole egg of the present invention | 0.8 g |

Production example 7 Health food
In 100 g of prescription example A (fine particles):

| | | |
|---|---|---|
| Defatted and powdered whole egg of the present invention | 45 | g |
| Lactose (200 M) | 35 | g |
| Corn starch | 15 | g |
| PVP (K-30) | 5 | g |

Fine particles were obtained using the above ingredients by a general method using a wet granulation method.

In 100 g of prescription example B (granules):

| | | |
|---|---|---|
| Defatted and powdered whole egg of the present invention | 33 | g |
| Lactose (200 M) | 44 | g |
| Corn starch | 18 | g |
| PVP (K-30) | 5 | g |

Granules were obtained using the above ingredients by a general method using an extruding granulation method.

Production example 8 Medical food
Prescription example A fluid diet (200 ml/pack)

| | |
|---|---|
| Defatted and powdered whole egg of the present invention | 2.6% |
| Malt dextrin | 39.0% |
| Casein Na | 13.0% |
| Vegetable oil | 12.0% |
| Vitamins | 1.0% |
| Minerals | 1.5% |
| Emulsifier | 0.2% |
| Lactoprotein | 10.3% |
| Phosphoric acid Na | 1.8% |
| Phosphoric acid K | 1.2% |
| Aromatic | 0.5% |
| Stabilizer (carrageenan) | 1.5% |
| Water | Residual |
| | quantity |

Prescription example B Drinkable preparation (soup type)

| | |
|---|---|
| Defatted and powdered whole egg of the present invention | 2.5% |
| Carrot (carrot paste) | 10.0% |
| Fresh cream | 12.0% |
| Lactose | 1.8% |
| Onion (onion extract) | 1.5% |
| Lactoprotein powder | 0.5% |
| Lactosucrose | 1.5% |
| Consommé powder | 0.5% |
| Wheat germ | 0.5% |
| Eggshell calcium | 0.2% |
| Milk serum calcium | 0.1% |
| Table salt | 0.2% |
| Emulsifier | 0.2% |
| Water | Residual |
| | quantity |

Production example 9 Chewing gum

| | | |
|---|---|---|
| Gum base | 200 | g |
| Sugar | 600 | g |
| Glucose | 80 | g |
| Starch syrup | 100 | g |
| Glycerine | 5 | g |
| Aromatic | 10 | g |
| Powdered whole egg of the present invention | 5 | g |

Production example 10 Milk pudding

| | |
|---|---|
| Skim milk | 5.0% |
| Sugar | 2.0% |
| Whole milk condensed milk | 10.0% |
| Liquid egg yolk of the present invention | 3.0% |
| Coconut oil | 3.0% |
| Table salt | 0.04% |
| Gelatinizer | 0.45% |
| Emulsifier | 0.1% |
| Water | 74.2% |
| Flavor | Proper quantity |
| Coloring matter | Proper quantity |

### INDUSTRIAL APPLICABILITY

According to the present invention, antibodies specific to digestive enzymes can be obtained at low cost and in large quantities. These antibodies can also be easily purified. Furthermore, from the above antibodies, the egg, and the processed product thereof containing such antibodies, an agent for inhibiting a digestive enzyme having a high degree of substrate specificity can be produced at low cost and with no toxicity. Besides, by the use of the egg and the processed product thereof of the present invention, foods having antiobesity action can also be produced at low cost. Furthermore, a composition containing the antibodies of the present invention that has been produced using 2 or more types of digestive enzymes as immunogens possesses a synergistic effect, so that it can be used as a very effective antiobesity agent. Based on the digestive enzyme-inhibiting effect of the antibodies of the present invention and the synergistic effect resulting from the action of each antibody, saccharide absorption-inhibiting action, action suppressing increases in blood glucose levels, anti-lipemic action, anti-arteriosclerosis action, action lowering blood triglyceride levels, and action lowering cholesterol levels can be provided.

1. A composition comprising an egg produced by a hen immunized with a digestive enzyme or a fragment thereof, or the processed product thereof, wherein the digestive enzyme comprises 2 or more types of digestive enzymes.

2. The composition of claim 1, which comprises an egg produced by the same hen immunized with 2 or more types of digestive enzymes or fragments thereof, or a processed product thereof.

3. The composition of claim 1, comprising a mixture of an egg produced by a hen immunized with at least 1 type of digestive enzyme or a fragment thereof, or the processed product thereof, and an egg produced by a hen immunized with a digestive enzyme differing from the digestive enzyme or a fragment thereof, or a processed product thereof.

4. The composition of any one of claims 1 to 3, wherein the digestive enzyme is selected from the group consisting of a glycolytic enzyme, a lipolytic enzyme, and a proteolytic enzyme.

5. The composition of any one of claims 1 to 4, wherein the processed product of the egg is an antibody.

6. An agent for inhibiting a digestive enzyme, which comprises the composition of any one of claims 1 to 5.

7. An antiobesity agent, which comprises the composition of any one of claims 1 to 5.

8. A food, which contains the composition of any one of claims 1 to 5.
